(19) United States
(12) Patent Application Publication
(10) Pub. No.: US 2004/0162586 A1
(43) Pub. Date: Aug. 19, 2004

(54) Defibrillator electrodes with identification tags

(76) Inventors: Kevin K. Covey, Marysville, WA (US); Larry R. Nygaard, Snohomish, WA (US)

Correspondence Address: INTELLECTUAL PROPERTY GROUP, FREDRIKSON & BYRON, P.A., 4000 PILLSBURY CENTER, 200 SOUTH SIXTH STREET, MINNEAPOLIS, MN, 55402, US

(21) Appl. No.: 10/423,685

(22) Filed: Apr. 25, 2003

Related U.S. Application Data
(60) Provisional application No. 60/448,348, filed on Feb. 18, 2003.

(51) Int. Cl. A61N 1/39

(52) U.S. Cl. 607/5

(57) ABSTRACT

Electrodes having electrode identification tags are used on medical devices, particularly on defibrillators. One such defibrillator uses electrical devices to query the tags and acquire electrode identification information. Software is also incorporated for querying the tags automatically and interpreting the acquired information.

### CROSS-REFERENCE TO RELATED APPLICATION

This patent incorporates by reference and claims priority to the entire contents of U.S. Provisional Application Serial No. 60/448,348, filed Feb. 18, 2003.

### TECHNICAL FIELD

The invention is related to the field of medical devices, and in particular, to devices, software, and methods for identifying electrodes utilized with defibrillators.

### BACKGROUND OF THE INVENTION

A sudden cardiac arrest (SCA) incident can kill a victim. More aptly called sudden cardiac death, SCA is a condition in which the heartbeat stops suddenly and unexpectedly. It is caused by life-threatening arrhythmias, which are abnormalities in the heart's electrical system. The most common arrhythmia is ventricular fibrillation. In this condition, the heart beats too chaotically to be effective in pumping blood to the body and brain.

SCA is one of the leading causes of death among American adults: it kills approximately 225,000 people a year. Two out of every three deaths involving SCA happen outside of the hospital. There is no predictability with SCA, as it can happen to anyone, anywhere—even to a child. Risk of SCA increases with age. Although pre-existing heart disease is a common cause of cardiac arrest, many victims are found to have never had any prior heart problems.

Without immediate treatment, an SCA victim almost always dies. In the initial few minutes of an SCA incident, the probability of survival for the victim decreases by 10% every minute. In order to increase chances of survival, treatment must be administered as soon as possible.

Responding effectively to such SCA incidents usually requires specially trained paramedics using specially designed equipment. The equipment includes a defibrillator, most specifically, an automated external defibrillator (AED), which is used to supply an electrical shock that stops the fibrillation.

The AED supplies a charge though the heart via electrodes. The electrodes of the defibrillator include a pair of wires that emanate from the defibrillator, and terminate in electrically conductive areas that are to be applied to the torso of the victim. The electrodes can generally be made to stick to a chest of a patient if used with adhesive.

Potential problems exist when AEDs are not used on a regular basis. The electrodes used with defibrillators come in different varieties that may be difficult to identify. In addition, if adhesive is used with the electrodes as described above, the adhesive may dry out and not be useful. Further, the electrodes may be over their expiration date prior to their use in the field. Thus, the potential exists for a user to be unaware of the type or condition of the electrodes until their actual use. Unfortunately, for patients receiving treatment, this delayed realization may hinder their chances of survival. Thus, in regard to using defibrillators, it is important to identify the electrodes and their functioning status prior to their use in medical emergencies to ensure that they will function as intended during such emergencies.

There have been attempts to alleviate the problem of identifying electrodes because of the many kinds of specific use electrodes now available. Unfortunately, the number of different electrode designs will most likely continue to increase in the future, and only further complicate matters. Currently, some electrodes used with defibrillators can be identified by their connector styles. Unfortunately, this type of electrode identification is totally dependent on the user being familiar with the connector styles. Thus, the possibility exists for the wrong therapy to be delivered to the patient based on the user erroneously identifying the connector style. Other electrodes are now designed to have physical lock-outs on the connectors in order to be less dependent on the knowledge of the user for identification and more focused on preventing delivery of the wrong therapy to the patient. However, in using connectors with physical lock-outs, the compatibility of such electrodes is limited.

Another problem common with AED electrodes is that when there is a malfunction with the system, the electrodes are generally discarded. In turn, if the malfunction involved the electrodes, all positive identification of the problem is also discarded in the process. This information, while useless to the user, may be invaluable to the manufacturer in designing more efficient electrodes in the future.

The prior art has attempted to solve some of these problems, specifically the problem of electrode identification, by means of connector styles, resistor networks, and IC chips. Thus far, all are less than satisfactory. As mentioned above, identification by means of connector styles is dependant on the user for accurate identification. The cost of using resistor networks is prohibitive given the large volumes that are common for electrodes, and is effective only for identifying the electrode type, but not the electrode manufacturing lot-code. Further, using programmable IC chips is also cost prohibitive.

### SUMMARY OF THE INVENTION

The present invention solves the problems of the prior art. Generally, the present invention provides electrodes that contain electrode identification tags. The invention also provides a defibrillator having devices and methods for querying the tags and identifying the electrodes. The invention further provides software for automatically querying the tags and identifying the electrodes.

### BRIEF DESCRIPTION OF THE DRAWINGS

The invention will become more readily apparent from the following description, which proceeds with reference to the drawings, in which:

FIG. 1 is a schematic perspective view of a defibrillator in accordance with certain embodiments of the invention;

FIG. 2 is a block diagram of the defibrillator of FIG. 1 in accordance with certain embodiments of the invention;

FIG. 3 is a flowchart illustrating the progression of steps in a self-diagnostic test for a defibrillator in accordance with a certain embodiment of the invention;

FIG. 4 is a flowchart illustrating the progression of steps in a self-diagnostic test for a defibrillator in accordance with an alternate embodiment of the invention;

FIG. 5 is a flowchart illustrating the progression of steps in a self-diagnostic test for a defibrillator in accordance with another alternate embodiment of the invention; and

FIG. 6 is a block diagram illustrating a status monitor in accordance with certain embodiments of the invention.

### DETAILED DESCRIPTION OF THE INVENTION

The following detailed description is to be read with reference to the drawings, in which like elements in different figures have like reference numerals. The drawings, which are not necessarily to scale, depict selected embodiments, but are not intended to limit the scope of the invention. It will be understood that many of the specific details of the device incorporating the system illustrated in the drawings could be changed or modified by one of ordinary skill in the art without departing significantly from the spirit of the invention. The electrodes and corresponding electrode identification tags are designed for use on devices such as defibrillators, however they may be used on other medical devices having electrodes.

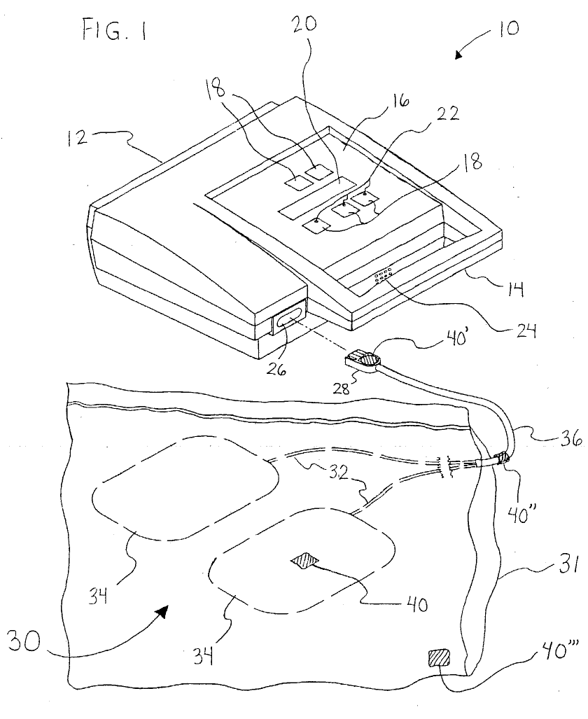

FIG. 1 shows a perspective view of a portable external defibrillator in accordance with certain embodiments of the invention. The defibrillator 10 preferably comprises an automated external defibrillator (AED). The defibrillator 10 is designed for use by a first responder during a medical emergency as described above. To facilitate use in the field, the defibrillator 10 is preferably formed with an impact resistant plastic housing 12 having an integral handle 14 to allow the user to easily carry the defibrillator 10 to a desired location. When used, the defibrillator 10 is preferably positioned so that a control panel 16 on the defibrillator 10 is oriented upwards towards the user. In certain preferable embodiments of the invention, buttons 18 on the control panel 16 provide for powering the defibrillator 10, analyzing the heart rhythms of the patient, and administering therapeutic shocks to the patient.

The defibrillator 10 provides instructions or other information to the user using one or more output elements. In certain embodiments of the invention, some of these output elements are located on the control panel 16. These may include one or more displays 20, e.g., liquid crystal displays (LCD), cathode ray tube (CRT) displays, etc., that present instructions to direct the user to perform different tasks, and one or more visual annunciators 22, e.g., light-emitting diodes (LEDs), strobe lights, etc., that illuminate or darken to convey status information. In conveying the status information of the defibrillator 10, the output elements may indicate, for example, whether an energy storage capacitor within the defibrillator 10 is sufficiently charged to provide a defibrillation pulse to a patient. The output elements may also include other output elements; for example, a speaker 24 integrally formed in the housing 12 of the defibrillator 10 could be used to provide a corresponding audible signal or spoken message to the user.

Located adjacent the handle 14 of the defibrillator 10 is a therapy/data port 26. The therapy/data port 26 is used to connect the defibrillator 10 to a patient (via electrodes), to an auxiliary component, or to a test load. Although many different connectors could be designed to mate with the therapy/data port 26, a QUIK-COMBO™ connector 28, sold by Medtronic Physio-Control Corporation of Redmond, Wash., is preferably used to mate with the port 26. The body of the therapy/data port 26 is formed of a non-conductive resilient material to mate with the connector 28. When mated with the connector 28, conductive receptacles (not shown) within the port 26 receive corresponding conductive pins (not shown) within the connector 28. The therapy/data port 26 is also formed with two integral seals (not shown) that extend around the body of the port 26 that prevent water, dirt, or other contaminants from entering the defibrillator 10.

When the connector 28 is used with a pair of electrodes 30 as shown, the corresponding connector 28 contains two conductive pins (not shown), each of which is connected to one of the electrodes 30 by a lead 32. The leads 32 are shown broken in their length since they are preferably of a length that is longer than what is represented. Although various types of electrodes may be used with the defibrillator 10, preferably the electrodes 30 are QUIK-COMBO™ pacing/defibrillation/ECG electrodes sold by Medtronic Physio-Control Corporation. QUIK-COMBO™ electrodes are disposable electrodes that are applied to the patient by peeling away a backing on the electrodes to expose an adhesive pad. To connect the electrodes 30 to the defibrillator 10, connector 28 is inserted into the therapy/data port 26. When not in use, the electrodes 30 are preferably kept within an airtight sealable pouch 31 stored next to the housing 12 of the defibrillator 10.

In use, the defibrillator 10 delivers one or more electrical shocks via the electrodes 30 to defibrillate the heart of a patient. An electrical source (not shown) that can generate the shocks is preferably located within the housing 12 of the defibrillator 10. As mentioned above, each of the electrodes 30 preferably comprises a conductor pad 34 having adhesive located thereon for its application. However, each of the pair of electrodes 30 may also comprise a conductor paddle having a handle that a user may grasp and hold while administering therapy to the patient. When mated with the port 26, the connector 28 couples the electrodes 30 to the electrical source of the defibrillator 10. In certain embodiments of the invention, an outer sheath 36 is utilized to cover a portion of the pair of leads 32 nearest the connector 28.

The defibrillator 10 may include a power supply (not shown) preferably located within the housing 12. The power supply for many models of the defibrillator 10 is a battery, although some models of the defibrillator 10 may be capable of being “line powered,” i.e., plugged into an electrical outlet. Battery power is advantageous in many respects. First, in many situations, the patient may be far from an electrical outlet. In those situations, the defibrillator 10 may rely upon a battery to supply energy to the electrical source for the defibrillation shocks. Second, using a battery enables the defibrillator 10 to be portable, and in turn, useful in a wider variety of emergency situations.

Preferably, the electrical source of the defibrillator 10 comprises an energy storage device (not shown), such as one or more capacitors, and a charging circuit (not shown), such as a flyback charger. When a defibrillation shock is needed, the charging circuit transfers energy from the power supply to the energy storage device. When the energy stored in the energy storage device reaches a desired level, the defibrillator 10 is ready to deliver electrical shock defibrillation therapy. In accordance with certain embodiments of the invention, the therapy is preferably delivered automatically using an AED, with conductor pads applied to the patient using adhesive as described above, however, the therapy could also be delivered manually using handheld conductor paddles instead.

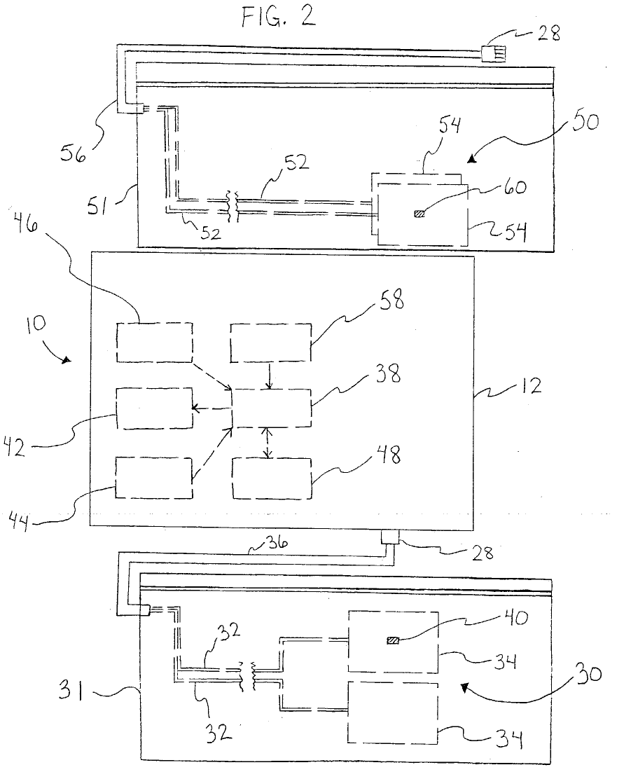

The defibrillator 10 further includes a processor 38 (not shown in FIG. 1, but represented in FIG. 2) mounted to the defibrillator 10, preferably within the housing 12. While only a single microprocessor is represented in FIG. 2, it will be appreciated that one or more processors may be mounted to the defibrillator 10 for redundancy, enhanced processing capability, or other functioning. The processor 38 is connected to the control panel 16 by a bus (not shown), and in turn, can receive commands generated by a first responder using the buttons 18 on the control panel 16. Additionally, voice data received on a microphone could be digitized and stored using techniques known in the art. To provide instructions or visual cues to the user, the processor 38 can transfer appropriate data via the bus to activate the display 20, the visual annunciators 22, or provide voiced commands over the speaker 24. The processor 38 preferably controls various functions of the defibrillator 10. For example, the processor 38 may govern charging of the energy storage device, evaluate heart rhythms of the patient sensed via the electrodes 30, or deliver the defibrillation shocks automatically.

Preferably, the processor 38 is also used to execute a routine that performs a self-diagnostic test. As a function of the self-diagnostic test, status information regarding the defibrillator 10 is acquired by the processor 38. The status information acquired during the test may pertain to the operating status of the defibrillator 10 and its attendant components. The information may include data indicative of whether the defibrillator 10 is in good working order, or conversely, data indicative of a failed or damaged component within the defibrillator 10. Data indicating that the battery is low, or that the battery is failing to hold a charge, may be examples of such a failed or damaged component within the defibrillator 10.

The status information acquired during the self-diagnostic test would also preferably include information in regard to the electrodes 30. The status information mentioned herein that pertains specifically to the electrodes 30 is referred to as electrode identification information. In accordance with certain embodiments of the invention, the electrode identification information for any one pair of electrodes 30 is preferably stored in a memory of a corresponding electrode identification tag 40. Such a tag 40 is preferably coupled to at least one of the pair of corresponding electrodes 30 for which the electrode identification information applies. As shown, there are at least three different locations on the pair of electrodes 30 which would provide adequate coupling surfaces for the tag 40. These locations include the conductor pads 34 (with tag 40), the electrode connector 28 (with tag 40′), and the outer sheath 36 covering the portion of the leads 32 (with tag 40″). Additionally, the tag 40 can be located on the pouch 31 (with tag 40′″) within which the electrodes 30 are stored when not in use. The tags 40, 40′, 40″, and 40′″ are similar in structure and function, and are only referenced differently (with primes) to indicate that all the tags represented are not the same tag. It should be assumed that the tags 40, 40′, 40″, and 40′″ are similar unless otherwise indicated.

The electrode identification information is preferably programmed in a memory of the tag 40, and such information includes one or optionally more of the electrode type, the electrode expiration date, the electrode manufacturing lot-code, and a distinguishing code. The distinguishing code is preferably a random 2 or 3 character alpha numeric code, and the purpose of including such a code would be to distinguish between two or more electrodes having the same manufacturing lot-codes and the same expiration dates. By distinguishing in this fashion, exact electrode identification can be provided to the manufacturer, which can be used for future development purposes in improving the electrodes 30.

In accordance with certain preferable embodiments of the invention, each electrode identification tag 40 is a Radio Frequency Identification (RFID) tag. RFID has become an important identification technology in applications such as inventory management, security access, personnel identification, factory automation, automotive toll debiting, and vehicle identification. In general, an RFID system would include an RFID transmitter-receiver unit used to query an RFID transponder or tag, which may be located at a distance from the transmitter-receiver unit. The RFID tag detects the interrogating or query signal and transmits a response signal containing encoded data back to the receiver. Information in the response signal may be encrypted, and a database of electrode identifying part numbers may be needed for cross-referencing the information in order to interpret the information.

RFID systems provide identification functions not found in other identification technologies such as optical indicia (e.g., bar code) recognition systems. For example, RFID systems may employ RFID tags containing read/write memory of several kilobytes or more. The RFID tags may be readable at a distance and do not require direct line-of-sight view by the reading apparatus (e.g., base station or interrogator). Further, several such RFID tags may be read by the RFID system at one time.

RFID tags may be entirely passive (i.e., having no power supply), which allows for availability in a small and portable package. However, this identification system would be only capable of operation over a relatively short range, limited by the size of an electromagnetic field used to supply power to the tags and to communicate with the tags. As an alternative, RFID tags may also utilize a larger active transponder device affixed to an object to be monitored which receives a signal from the interrogator. The device receives the signal, then generates and transmits a responsive signal. The interrogation signal and the responsive signal are typically radio-frequency (RF) signals produced by an RF transmitter circuit. Because active devices have their own power sources, they do not need to be in close proximity to an interrogator or reader to receive power via electromagnetic waves. As a consequence, active transponder devices tend to be more suitable for applications requiring tracking of a tagged device that may not be in close proximity to an interrogator. For example, active transponder devices tend to be more suitable for inventory control or tracking.

As shown in FIG. 2, many defibrillators additionally have a spare pair of electrodes 50, which, when not in use, are preferably kept within a spare airtight sealable pouch 51 stored next to the housing 12 of the defibrillator 10. Although various types of spare electrodes may be used with the defibrillator 10, preferably the spare electrodes 50 are QUIK-COMBO™ pacing/defibrillation/ECG electrodes sold by Medtronic Physio-Control Corporation. In the event of a malfunction of the pair of electrodes 30 originally intended for use with the defibrillator 10, or after the use and subsequent disposal of the pair of electrodes 30 originally intended for use with the defibrillator 10, the spare pair of electrodes 50 can be subsequently used as a replacement. The spare pair of electrodes 50 may be structurally and functionally equivalent to the original pair of electrodes 30, however, the spare pair of electrodes 50 may have been manufactured at a different time. Leads 52 of the spare pair of electrodes 50 are shown broken in their length since they are preferably of a length that is longer than what is represented.

In accordance with certain embodiments of the invention, the status information described above that is acquired from the self-diagnostic test would also preferably include spare electrode identification information. Just as in the case with the pair of electrodes 30, the spare electrode identification information for any one spare pair of electrodes 50 is preferably stored in a memory of a corresponding spare electrode identification tag 60. Further, each spare electrode identification tag 60 is preferably coupled to at least one of the corresponding spare pair of electrodes 50 for which the spare electrode identification information applies. Like the pair of electrodes 30, there are at least three different locations on the spare pair of electrodes 50 which would provide adequate coupling surfaces for the tag 60. These locations include conductor pads 54 (one of which is shown with tag 60), the electrode connector 28, and an outer sheath 56 covering the portion of the leads 52. Additionally, the tag 60 can be located on the pouch 51 within which the electrodes 50 are stored when not in use. No matter where located, the tag 60 would be not vary in structure and function.

The tag 60 is preferably programmed with the spare electrode identification information, and such information includes one or optionally more of a spare electrode type, a spare electrode expiration date, a spare electrode manufacturing lot-code, and the distinguishing code. Once again, the distinguishing code is preferably a random 2 or 3 character alpha numeric code. The purpose of including the code would again be to distinguish one or more same type electrodes having same manufacturing lot-codes and same expiration dates. By distinguishing in this fashion, exact electrode identification can be provided to the manufacturer, which can be used for future development purposes in improving the electrodes 50.

A block diagram depicting the system schematic of the invention is shown in FIG. 2. The system components include the defibrillator 10, the pair of electrodes 30 originally intended for use with the defibrillator 10 (shown in the sealable pouch 31), and the spare pair of electrodes 50 (shown in the spare sealable pouch 51). At least one of the pair of electrodes 30 originally intended for use with the defibrillator 10 has a single electrode identification tag 40 preferably mounted to one of the conductor pads 34. Likewise, at least one of the spare pair of electrodes 50 has a single spare electrode identification tag 60 preferably mounted to one of the conductor pads 54. In accordance with certain embodiments of the invention, electrical components including the processor 38 with memory 48, a transmitter 42, a primary receiver 44, and a spare receiver 46 are mounted to the defibrillator 10, preferably within the housing 12. The arrows shown connecting the electrical components illustrate the direction of outgoing signals from each component, as to whether the signals are being sent to the processor 38, or whether the signals are being sent from the processor 38. The transmitter 42 receives signals from the processor 38. Conversely, the primary receiver 44 and the spare receiver 46 transmit signals to the processor 38. The memory 48, while represented as separate to the processor 38, is preferably integral with the processor 38. The memory 48 is represented separate to the processor 38 to demonstrate that it both sends signals to and transfers signals from the processor 38. Of course, other components are connected to the processor 38 to aid in its function, as is well-known in the art; the components shown in FIG. 2 are merely those relevant to the preferred embodiment. Suitable equivalents may be substituted as well.

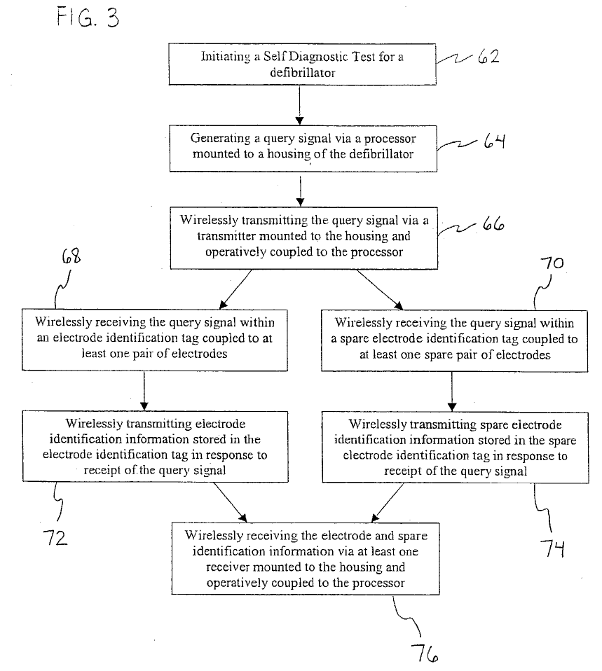

FIGS. 3, 4, and 5 illustrate flow charts showing operations of the self-diagnostic test for a defibrillator in accordance with certain embodiments of the invention. As described above, the self-diagnostic test of the defibrillator 10 is run in order to acquire status information pertaining to the operating status of the defibrillator 10 and its attendant components. As also mentioned above, in accordance with the invention, the status information additionally includes information regarding both pairs of electrodes 30 and 50, namely their electrode identification information. While FIGS. 3, 4, and 5 depict steps of the self-diagnostic test, the steps are limited in scope to only include acquiring the status information for the electrodes 30 and 50. While the flowcharts are limited in this regard, the invention should not be limited as such. In addition, while FIGS. 3, 4, and 5 illustrate preferred embodiments in regard to the self-diagnostic test steps affecting the electrodes 30 and 50, it should be recognized that steps in any of the flow charts could be exchanged or even in some cases eliminated without diverting from the spirit the invention.

As shown and described above, FIG. 2 illustrates a block diagram of the invention in accordance with both flowcharts, depicting each of the system components mentioned in the flowcharts and the proximity of the components within the defibrillation system. Based on the functions described below for each of the components within the flowcharts, it is contemplated that those skilled in the art would find it obvious to be able to select appropriate devices and corresponding manufacturers for each component.

FIG. 3 shows a flowchart illustrating the steps of a certain embodiment of the self-diagnostic test. The initial step 62 involves an initiation of the self-diagnostic test for the defibrillator 10. Preferably, this is a function of the processor 38 mounted to the defibrillator 10. The processor 38 may have a timing mechanism to specifically initiate the test on a periodic basis, e.g., once a week, to check and log the status information of the electrodes 30 and 50. Alternatively, the processor 38 may only initiate the test when the defibrillator 10 is powered, or in response to a detected problem in a prior test. It is also contemplated that the self-diagnostic test could be initiated by an external signal to the processor 38. This external signal could originate from a handheld remote control module, or a central processing unit that is in remote communication with the processor 38.

Upon initiation of the self-diagnostic test of the defibrillator 10, the next step 64 involves the processor 38 generating a query signal that is used to interrogate the electrode identification tags. In order to facilitate this interrogation, the processor 38 sends the query signal to the transmitter 42 that is operatively coupled to the processor 38. A subsequent step 66 entails the transmitter 42 wirelessly transmitting the query signal, preferably by radio waves.

In accordance with certain embodiments of the invention, steps 68 and 70 involve the transmitted query signal being wirelessly received by at least two electrode identification tags. Preferably, these tags at least include the electrode identification tag 40 that is coupled to the pair of electrodes 30, along with the spare electrode identification tag 60 that is coupled to the spare pair of electrodes 50. These tags 40 and 60 are preferably RFID tags. The tags 40 and 60 are preferably adapted to wirelessly receive the query signal. The tags 40 and 60 are also preferably adapted to wirelessly transmit the electrode identification information upon their receipt of the query signal. In response to the query signal being received by the tag 40 coupled to the pair of electrodes 30 in step 68, step 72 includes the electrode identification information corresponding to the electrodes 30 and stored in memory within the tag 40 being wirelessly transmitted, preferably by radio waves. Likewise, in response to the query signal being received by the tag 60 coupled to the spare pair of electrodes 50 in step 70, step 74 includes the spare electrode identification information corresponding to the spare electrodes 50 and stored in memory within the tag 60 being wirelessly transmitted, preferably by radio waves.

The electrode identification information, transmitted from each of the RFID tags 40 and 60 in respective steps 72 and 74, is wirelessly received by at least one receiver, preferably by radio waves, in step 76. The at least one receiver is mounted to the housing 12 of the defibrillator 10. In certain embodiments of the invention, the electrode identification information transmitted from the tag 40 will be received by a primary receiver 44, while the spare electrode identification information transmitted from the spare tag 60 will be received by a spare receiver 46. This is accomplished by selecting appropriate receiving ranges for both of the receivers. Preferably, each of the receivers 44 and 46 would also be adapted to distinguish, differentiate, or filter signals other than what they are intended to receive. In certain preferable embodiments of the invention, the electrode identification information transmitted from both tags 40 and 60 will be received by the primary receiver 44, thereby eliminating a need for the spare receiver 46. The at least one receiver receiving the electrode identification information is operatively coupled to the processor 38. As such, the information received by the at least one receiver can be transferred to the processor 38, and the information can then be logged, i.e., stored in the memory 48 to the processor 38. The processor 38 would preferably be adapted to segregate the electrode identification information transmitted from the tag 40 from the spare electrode identification information transmitted from the tag 60.

In certain embodiments of the invention, the electrode identification information received by the at least one receiver in step 76 may further be interpreted using software and a database of identifying information, which would be incorporated within the processor 38. Using the software, the electrode identification information would be cross-referenced with the information in the database, preferably held in the memory 48 to the processor 38. In turn, the electrode identification information could be interpreted, and based on the interpretation, messages or indications would preferably be communicated to the user via the output elements (i.e., the displays 20, the annunciators 22, the speaker 24, etc.) as to the identity and condition of the electrodes 30 and 50. As to the identity and condition of the electrodes 30 and 50, the user could be notified, for example, whether the electrodes 30 and 50 are designed for pediatric therapy or adult therapy and whether the electrodes 30 and 50 are past their corresponding expiration dates. This communication with the user would be facilitated by the processor 38 being coupled to the control panel 16 (FIG. 1) via the bus.

Additionally represented in the block diagram of FIG. 2 is a sensor 58 mounted to the housing 12 of the defibrillator 10. The sensor 58 is adapted to measure environmental conditions proximate to the defibrillator 10. Preferably, the environmental conditions measured would include air temperature and air humidity. The sensor 58 would be operatively coupled to the processor 38, such that the measured environmental conditions could be transferred to the processor 38. Once received by the processor 38, the measured environmental conditions could be logged, or stored in the memory 48 to the processor 38. The sensor 58, and corresponding measured environmental conditions, could, in turn, be utilized to evaluate the status information in regard to the electrodes 30 and 50. For example, if the environmental conditions, such as the air temperature and air humidity mentioned above, exceed normal conditions for long durations, it is likely that such conditions would have a detrimental effect on the life of the electrodes 30 and 50. As a consequence, the expiration dates of the electrodes would need to be interpreted and subsequently evaluated taking the environmental conditions into account. While it is described that the measured environmental conditions would be utilized for evaluating the state of the electrodes, it is also contemplated that the environmental conditions could likewise be used for evaluating the state of the defibrillator 10 and its attendant components. For example, such adverse environmental conditions described above could affect the life of the defibrillator battery, as well as other electrical components mounted to the defibrillator 10.

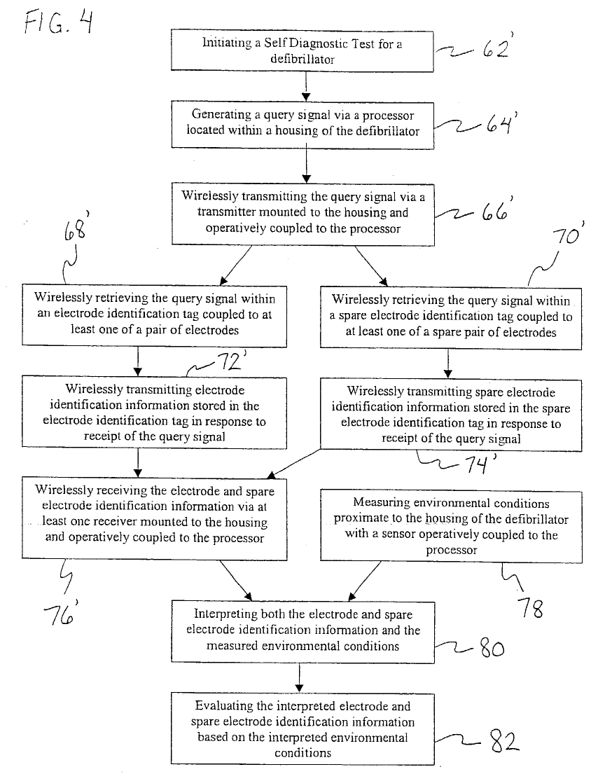

FIG. 4, like FIG. 3, shows a flowchart illustrating the steps of an alternate embodiment of the self-diagnostic test. As illustrated, the steps 62 through 76 are carried over from FIG. 3, and are represented in FIG. 4 as steps 62′ through 76′. It should be assumed that the steps 62′ through 76′ from FIG. 4 are similar to the steps 62 through 76 from FIG. 3 unless otherwise indicated. As such, these steps need not be discussed again as they have already been described above. However, during steps 62 through 76 of the self-diagnostic test, the sensor 58 is preferably measuring environmental conditions proximate to the housing of the defibrillator 10 in step 78. In particular, as the electrode identification information is being acquired by the processor 38, i.e., in steps 62 through 76, the environmental conditions are being measured by the sensor 58 and subsequently being transferred to the processor 38. In certain embodiments of the invention, these transferred environmental conditions can then be logged, i.e., stored in the memory 48 to the processor 38.

In certain embodiments of the invention, the electrode identification information and the measured environmental conditions received by the processor 38, are preferably interpreted in step 80, using software and a database of identifying information, which would be incorporated within the processor 38. Using the software, the electrode identification information would be cross-referenced with the information in the database, preferably held in the memory 48 to the processor 38. Using the software, the environmental conditions, whether using measured data from the instant self-diagnostic test or encompassing several sets of measured data (some of which had been stored in the memory 48 to the processor 38), would be interpreted as to whether it represented severe or adverse conditions to the electrodes 30 and 50. In turn, the software would be further used to evaluate the interpreted electrode identification information based on the interpreted environmental conditions in step 82.

As a consequence of the evaluation described above, the software could be additionally used for adjusting the expiration dates of the electrodes 30 and 50 if warranted by the interpreted environmental conditions. Based on the evaluation, messages or indications would be preferably communicated to the user via the output elements (i.e., the displays 20, the annunciators 22, the speaker 24, etc.) as to the identity and condition of the electrodes 30 and 50. As to the identity and condition of the electrodes 30 and 50, the user could be notified, for example, whether the electrodes 30 and 50, are designed for pediatric therapy or adult therapy and whether the electrodes 30 and 50 are past their corresponding expiration dates, using the adjusted values of each if adjusting had been warranted in using the software. This communication with the user is facilitated by the processor 38 being coupled to the control panel 16 (FIG. 1) via the bus.

While the sensor 58 is shown as being mounted to the housing 12 of the defibrillator 10 in FIG. 2, in certain preferable embodiments of the invention, one sensor 58 is instead adapted to each of the electrode identification tags 40 and 60. As such, when the tags 40 and 60 are queried, the tags 40 and 60 not only transmit the corresponding programmed electrode identification information in response, but also the measured environmental conditions proximate to each pair of electrodes 30 and 50. The environmental conditions preferably would include air temperature and air humidity, and would be measured by each corresponding sensor 58 adapted in each tag 40 and 60.

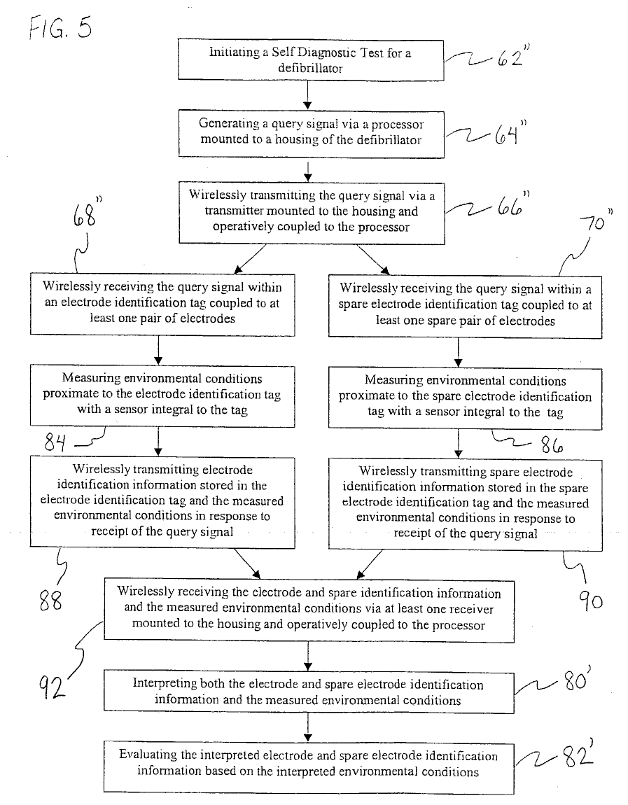

FIG. 5, like FIGS. 3 and 4, shows a flowchart illustrating the steps of another embodiment of the self-diagnostic test. As illustrated, the steps 62 through 70 are carried over from FIG. 3, and are represented in FIG. 5 as steps 62″ through 70″. In addition, the steps 80 and 82 are carried over from FIG. 4, and are represented in FIG. 5 as steps 80′ and 82′. It should be assumed that the steps 62″ through 70″ from FIG. 5 are similar to the steps 62 through 70 from FIG. 3 unless otherwise indicated. Further, it should also be assumed that the steps 80′ and 82′ from FIG. 5 are similar to the steps 80 and 82 from FIG. 4 unless otherwise indicated. As such, these steps need not be discussed again as they have already been described above. However, during steps 84 and 86, the environmental conditions proximate to each tag 40 and 60 are respectively measured by the corresponding sensors 58. In response to the query signal being received by the tag 40 coupled to the pair of electrodes 30 in step 68, step 88 includes the electrode identification information corresponding to the electrodes 30 (and stored in the memory within the tag 40), along with the measured environmental conditions by the sensor 58 within the tag 40, being wirelessly transmitted, preferably by radio waves. Likewise, in response to the query signal being received by the tag 60 coupled to the spare pair of electrodes 50 in step 70, step 90 includes the spare electrode identification information corresponding to the spare electrodes 50 (and stored in the memory within the tag 60), along with the measured environmental conditions by the sensor 58 within the tag 60, being wirelessly transmitted, preferably by radio waves.

The electrode identification information and environmental conditions, transmitted from each of the tags 40 and 60 in respective steps 88 and 90, are wirelessly received by at least one receiver, preferably by radio waves, in step 92. The at least one receiver is mounted to the housing 12 of the defibrillator 10. In certain embodiments of the invention, the electrode identification information and environmental conditions transmitted from the tag 40 will be received by the primary receiver 44, while the spare electrode identification information and environmental conditions transmitted from the spare tag 60 will be received by the spare receiver 46. This is accomplished by selecting appropriate receiving ranges for both of the receivers. In certain preferable embodiments of the invention, the electrode identification information and environmental conditions transmitted from both tags 40 and 60 will be received by the primary receiver 44, thereby eliminating a need for the spare receiver 46. The at least one receiver receiving the electrode identification information and environmental conditions is operatively coupled to the processor 38. As such, the information received by the at least one receiver can be transferred to the processor 38, and the information can then be logged, i.e., stored in the memory 48 to the processor 38. The processor 38 would preferably be adapted to segregate the electrode identification information and environmental conditions transmitted from the tag 40 from the spare electrode identification information and environmental conditions transmitted from the tag 60.

Industry standards exist that define procedures for determining shelf-life, and in turn, the expiration date, for electrodes. These standards generally involve testing the electrodes at nominal temperatures and humidity values for specific amounts of time from the date of manufacturing. Unfortunately, these standards do not take into consideration the actual environment that the electrodes may be exposed to in the field.

As described above, if the actual environment conditions are worse (more severe) than nominal values, the electrodes could be adversely affected, and could expire sooner than what had been nominally designated. Alternatively, if the actual environment conditions are better (less severe) than nominal values, the electrodes may not be affected as estimated, and could actually have later expiration dates than what had been nominally designated. As such, the electrodes are either being relied upon after the electrodes have lapsed past their effective expiration dates, or the electrodes are being discarded prematurely because they have gone over their initially designated expiration dates.

In order to evaluate an expiration date on an electrode, and be able to accurately adjust the date based on the environmental conditions (e.g., air temperature and air humidity) proximate to the electrodes, the electrode must be initially profiled. This profiling involves performance testing on the electrodes, in regard to testing the effects of temperature vs. time and humidity vs. time on the electrodes. The profiling will include subjecting the electrodes to clinical therapy efficacy testing, clinical diagnostic efficacy testing, clinical patient outcome efficacy testing, as well as electrical bench testing and mechanical adhesive testing.

The temperature segment of the electrode profile will be created by subjecting the electrode to various temperatures until a time at which the electrode fails. From the data obtained, a curve can be extrapolated to show the life of the electrode based on the temperature (i.e., approximately when the electrodes will fail based on a given air temperature proximate to the electrode).

The humidity segment of the electrode profile will be created by subjecting electrodes to moisture vapor transmission testing (MVTR) for a set period of time at different humidity and temperature ranges. Testing samples would preferably include packaged and non-packaged electrodes following their time of manufacture. From the data obtained, a curve can be extrapolated to show the amount of time necessary for moisture to be driven out of (or into) the electrode at different temperatures and humidity values.

In accordance with certain embodiments of the invention, the processor 38 in the defibrillator 10 is programmed to include a “Von't Hoff” curve for accelerated aging, as well as the temperature and humidity profiles described above. Base data including worst case shipping or storage profiles and nominal temperature and humidity profiles is also preferably programmed as well.

In use, when the defibrillator 10 queries an electrode 30 for the first time, the defibrillator 10 via the programmer 38 will log the electrode identification information transmitted as well as the measured environmental conditions. Upon logging the information, a “shelf life” profile for that electrode 30 will be started. The processor 38 inputs the measured environmental conditions (i.e., temperature and humidity) into the programmed curves, and calculates the shelf-life for the electrode 30. As more data is available (i.e., as the electrodes are queried more), a more accurate prediction of shelf-life is obtained, which can be used to adjust the designated expiration date for the electrodes. This adjusted expiration date can further be communicated to the user through the output elements on the defibrillator 10.

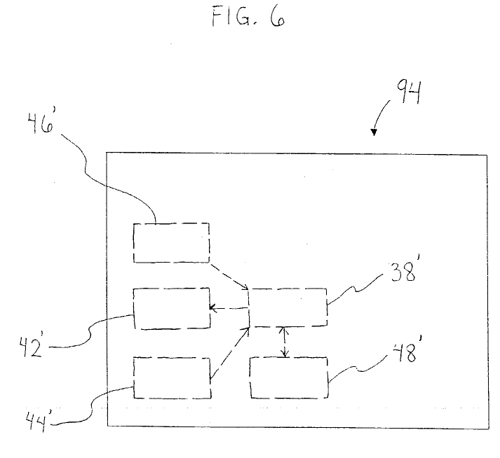

In certain embodiments of the invention, there may also exist a status monitor 94, represented in FIG. 6. Such a status monitor 94 could be a handheld module or a external central processing unit, which is remote from the defibrillator 10 but is in communication with the electrode identification tags 40 and 60 on the respective electrodes 30 and 50. With this communication with the tags 40 and 60, the status monitor 94 may wirelessly transmit the query signal for the status information of the electrodes 30 and 50 to the respective tags 40 and 60. In this scenario, the query signal sent from the status monitor 94 would be external to the defibrillator 10, and more specifically, would not involve the processor 38 of the defibrillator 10. Thus, the self-diagnostic test would not be initiated, however, the tags 40 and 60 would still be interrogated by the outside query signal. In turn, the status information of the electrodes 30 and 50, or the electrode identification information, would be wirelessly transmitted by the tags 40 and 60 to the status monitor 94.

A block diagram depicting the status monitor 94 is shown in FIG. 6. Since the status monitor 94 would preferably interrogate the electrodes 30 and 50 in a fashion similar to that of the defibrillator 10, the status monitor preferably includes similar electrical components for doing so. These electrical components include a processor 38′ with memory 48′, a transmitter 42′, a primary receiver 44′, and a spare receiver 46′, all of which are mounted to the status monitor 94. As illustrated, the electrical components 38, 42, 44, 46, and 48 are carried over from FIG. 2, and are represented in FIG. 6 as electrical components 38′, 42′, 44′, 46′, and 48′. It should be assumed that the components 38′, 42′, 44′, 46′, and 48′ from FIG. 6 are similar in structure and function to the electrical components 38, 42, 44, 46, and 48 from FIG. 2 unless otherwise indicated. As such, these components need not be described again as they have already been described above. Of course, other components are connected to the processor 38′ to aid in its function, as is well-known in the art; the components shown in FIG. 6 are merely those relevant to the preferred embodiment. Suitable equivalents may be substituted as well.

In certain embodiments of the invention, the communication between the status monitor 94 and the tags 40 and 60 is preferably done over a network. In particular, the tags 40 and 60 include a communication interface that establishes a communication link with a communication interface in the status monitor 94 over the network. As such, query signals from the status monitor 94 and responses from the tags 40 and 60 can be communicated over the network. The network may be any network. The network may comprise, for example, a public switched telephone network, a cellular telephone network, a local area network, a wide area network, a global computer network such as the Internet, an integrated services digital network, or the like. In some venues in which the electrode 30 and 50 may be deployed, the venue may include a dedicated security network or a private building maintenance network. Either may serve as the network. The network may include hard-wired electrical or optical communication links, wireless links, or a combination of both.

As described, the status monitor 94 may interrogate the tags 40 and 60 by transmitting the query signal for status information. The status monitor 94 may initiate the interrogation in response to a command from a responsible person, or the status monitor 94 may initiate the query signal automatically. Automatically transmitting a query signal may be part of a routine periodic interrogation, or the automatic interrogation may be in response to prior status information received from the tags 40 and 60 or from other tags being queried.

When the status monitor 94 receives the electrode status information, the status monitor 94 may update a status log. The status log, which may be stored in a memory of the status monitor 94, may include status information pertaining to the readiness of the electrodes 30 and 50. The status log may also record corrective measures that are indicated or that have been taken. In this way, the status monitor 94 helps the responsible person maintain a status log showing the status and repair history of the devices in the system. The logged status information may include, for example, a summary of the electrodes' location and operational status, such as “ready,” “out of service,” “door open” or “in use.” In certain embodiments of the invention, when the status information received in response to the interrogation indicates a matter requiring prompt attention, the status monitor 94 may wirelessly transmit a signal to the defibrillator 10, generating a response (e.g., warnings on displays 20, flashing lights from the annunciators 22, or audible noise over the speaker 24) to notify the user that corrective action may be required.

The status monitor 94 or the above-mentioned handheld portable module may wirelessly transmit one or more interrogations to electrode identification tags on one or more defibrillators and may receive status information in response from each of the tags. In contrast, as mentioned above, one or more processors, each mounted on a corresponding defibrillator, may receive the external signal described above for initiation of the self-diagnostic test, and in response, each processor would wirelessly transmit the status information to the source of the external signal, or another designated device that has been programmed within the memory to the processor. Additionally, the defibrillators may automatically transmit the status information wirelessly in regard to a programmed timetable, e.g., once a week.

Advantageously, the invention is not limited to any particular system. Rather, the invention may be practiced with systems of limitless configurations. Any number of electrodes may be tracked and monitored with the invention. The invention is not limited to systems in which defibrillators are transportable. In some instances, it may be beneficial to deploy the defibrillators in a fixed located. Alternatively, the defibrillator 10 could be deployed in a fixed location, but on a movable vehicle such as an ambulance or a vehicle used by a security guard.

Many examples of communication techniques may be contemplated, and the invention should not be limited to the techniques explicitly described. Communication may be based upon optical communication links, magnetic communication links, infrared communication links, or visual status change detectors. Furthermore, radio frequency has been described, but the invention is not limited to the techniques explicitly described. A cellular telephone link, for example, may employ any recognized communication protocol, such as code division multiple access (CDMA), Global System for Mobile Communications (GSM), or General Packet Radio Service (GPRS).

Moreover, the invention includes software to carry out many of the techniques described herein. The invention may be embodied as a computer-readable medium that includes instructions for causing a programmable processor to carry out the methods described above. A “computer-readable medium” includes but is not limited to read-only memory, flash memory and a magnetic or optical storage medium. The instructions may be implemented as one or more software modules, which may be executed by themselves or in combination with other software. The instructions and the media are not necessarily associated with any particular processor or other apparatus, but may be carried out by various general-purpose or specialized machines. The instructions may be distributed among two or more media and may be executed by two or more machines. The machines may be coupled to one another directly, or may be coupled through a network.

While a preferred embodiment of the present invention has been described, it should be understood that various changes, adaptations, and modifications may be made therein without departing from the spirit of the invention and the scope of the appended claims.

What is claimed is:

1. A defibrillator comprising:
a housing having an electrical source adapted to generate a defibrillation shock;
a pair of electrodes adapted to be operatively coupled to the electrical source and adapted to deliver the defibrillation shock to a patient;
an electrode identification tag coupled proximate to at least one of the electrodes and adapted to wirelessly transmit electrode identification information; and
a primary receiver mounted to the housing and adapted to wirelessly receive the transmitted electrode identification information.

2. The defibrillator of claim 1, wherein the defibrillator is an automated external defibrillator.

3. The defibrillator of claim 1, wherein the pair of electrodes includes a connector for coupling the electrodes to the electrical source, the tag being coupled to the connector.

4. The defibrillator of claim 1, wherein the pair of electrodes includes an outer sheath covering a portion of a pair of leads each coupling a connector to one of the electrodes, the tag being coupled to the outer sheath.

5. The defibrillator of claim 1, wherein each of the pair of electrodes comprises a conductor pad, the tag being coupled to one of the conductor pads.

6. The defibrillator of claim 1, further comprising a pouch adapted to contain the electrodes when the electrodes are not in use, the tag being coupled to the pouch.

7. The defibrillator of claim 1, wherein the electrode identification information includes one or optionally more of an electrode type, an electrode expiration date, an electrode manufacturing lot-code, and a distinguishing code.

8. The defibrillator of claim 7, wherein the distinguishing code is adapted to distinguish one or more electrodes having same manufacturing lot-codes and same expiration dates.

9. The defibrillator of claim 1, wherein the tag comprises memory for storage of the electrode identification information.

10. The defibrillator of claim 1, wherein the tag is adapted to wirelessly transmit the electrode identification information upon its receipt of a query signal.

11. The defibrillator of claim 10, wherein the tag includes a transponder adapted to wirelessly transmit the electrode identification information upon its receipt of a query signal.

12. The defibrillator of claim 10, wherein the housing includes a transmitter adapted to wirelessly transmit the query signal.

13. The defibrillator of claim 12, wherein the transmitter is adapted to wirelessly transmit the query signal to acquire the electrode identification information as part of a defibrillator self-diagnostic routine.

14. The defibrillator of claim 1, wherein the at least one tag is adapted to wirelessly transmit the electrode identification information via radio waves.

15. The defibrillator of claim 1, wherein the pair of electrodes are located within sufficient range of the housing for the primary receiver to wirelessly receive the electrode identification information.

16. The defibrillator of claim 1, wherein the pair of electrodes comprises a spare pair of electrodes and the electrode identification tag comprises a spare electrode identification tag adapted to wirelessly transmit spare electrode identification information.

17. The defibrillator of claim 16, wherein the spare electrode identification information includes one or optionally more of a spare electrode type, a spare electrode expiration date, a spare electrode manufacturing lot-code, and a distinguishing code.

18. The defibrillator of claim 16, wherein the housing has a spare receiver adapted to wirelessly receive the spare electrode identification information.

19. The defibrillator of claim 18, wherein the housing includes a processor coupled to the spare receiver and the primary receiver, the processor adapted to interpret the electrode identification information from the primary receiver and the spare electrode identification information from the spare receiver.

20. The defibrillator of claim 16, wherein the primary receiver is adapted to wirelessly receive the spare electrode identification information.

21. The defibrillator of claim 20, wherein the housing includes a processor coupled to the primary receiver, the processor adapted to interpret the electrode identification information and the spare electrode identification information from the primary receiver.

22. The defibrillator of claim 21, wherein the processor is operatively coupled to at least one output element that is adapted to communicate the interpreted electrode identification information to a user of the defibrillator.

23. The defibrillator of claim 22, wherein the at least one output element comprises a series of light emitting diodes.

24. The defibrillator of claim 1, further including at least one sensor adapted to measure environmental conditions.

25. The defibrillator of claim 24, wherein the at least one sensor is coupled to the tag and adapted to measure environmental conditions proximate to the tag.

26. The defibrillator of claim 24, wherein the at least one sensor is coupled to the housing and adapted to measure environmental conditions proximate to the tag.

27. The defibrillator of claim 24, wherein the environmental conditions include air temperature and air humidity.

28. The defibrillator of claim 24, further comprising a processor mounted to the housing.

29. The defibrillator of claim 28, wherein the at least one sensor is operatively coupled to the processor.

30. The defibrillator of claim 28, wherein the processor is adapted to interpret the electrode identification information and the measured environmental conditions.

31. The defibrillator of claim 30, wherein the processor is adapted to evaluate the interpreted electrode identification information based on the interpreted environmental conditions.

32. The defibrillator of claim 31, wherein the processor is operatively coupled to at least one output element adapted to communicate the evaluated electrode identification information to a user of the defibrillator.

33. A device comprising:
a pair of electrodes adapted to deliver a defibrillation shock to a patient when operatively coupled to an electrical source; and
an electrode identification tag coupled proximate to at least one of the electrodes and adapted to wirelessly receive a query signal.

34. The device of claim 33, wherein the electrical source comprises an automated external defibrillator.

35. The device of claim 33, wherein the pair of electrodes includes a connector for coupling the electrodes to the electrical source, the tag being coupled to the connector.

36. The device of claim 33, wherein the pair of electrodes includes an outer sheath covering a portion of a pair of leads each coupling a connector to one of the electrodes, the tag being coupled to the outer sheath.

37. The device of claim 33, wherein each of the pair of electrodes comprises a conductor pad, the tag being coupled to one of the conductor pads.

38. The device of claim 33, further comprising a pouch adapted to contain the electrodes when the electrodes are not in use, the tag being coupled to the pouch.

39. The device of claim 33, wherein the tag is adapted to wirelessly transmit electrode identification information upon its receipt of the query signal.

40. The device of claim 39, wherein the tag includes a transponder adapted to wirelessly transmit the electrode identification information upon its receipt of the query signal.

41. The device of claim 39, wherein the tag is adapted to wirelessly transmit the electrode identification information via radio waves.

42. The device of claim 39, wherein the tag comprises memory for storage of the electrode identification information.

43. The device of claim 39, wherein the electrode identification information includes one or optionally more of an electrode type, an electrode expiration date, an electrode manufacturing lot-code, and a distinguishing code.

44. The device of claim 43, wherein the distinguishing code is adapted to distinguish one or more electrodes having same manufacturing lot-codes and same expiration dates.

45. A method of identifying electrodes associated with a defibrillator, comprising:
wirelessly transmitting a query signal from a housing, the housing having an electrical source adapted to generate a defibrillation shock, the electrodes selectively delivering the defibrillation shock to a patient when coupled to the electrical source;
wirelessly receiving the query signal within an electrode identification tag coupled proximate to at least one of the electrodes;
wirelessly transmitting electrode identification information stored in the electrode identification tag in response to receipt of the query signal; and
wirelessly receiving the electrode identification information within the housing.

46. The method of claim 45, further comprising communicating the information through an output element.

47. The method of claim 45, further comprising wirelessly transmitting spare electrode identification information stored in a spare electrode identification tag associated with a spare pair of electrodes in response to receipt of the query signal.

48. The method of claim 45, further comprising measuring environmental conditions by at least one sensor.

49. The method of claim 48, further comprising evaluating the electrode identification information based on the environmental conditions.

50. A defibrillator comprising:
means for wirelessly transmitting a query signal from a housing, the housing having an electrical source adapted to generate a defibrillation shock,
electrode means selectively delivering the defibrillation shock to a patient when operatively coupled to the electrical source,
means for wirelessly receiving the query signal within an electrode identification tag coupled proximate to the electrode means,
means for wirelessly transmitting electrode identification information stored in the electrode identification tag in response to receipt of the query signal, and
means for wirelessly receiving the electrode identification information within the housing.

51. The defibrillator of claim 50, further comprising means for communicating the electrode identification information to an output element.

52. The defibrillator of claim 50, further comprising means for sensing environmental conditions.

53. The defibrillator of claim 52, further comprising means for evaluating the electrode identification information based on the environmental conditions.
